(19) United States
(12) Patent Application Publication
(10) Pub. No.: US 2026/0047832 A1
(43) Pub. Date: Feb. 19, 2026

(54) TEXTURED MEMBERS OF MEDICAL DEVICES

(71) Applicant: Boston Scientific Scimed, Inc., Maple Grove, MN (US)

(72) Inventors: Scott E. BRECHBIEL, Johnston, RI (US); Scott Edward CORBEIL, Litchfield, NH (US); Ryan V. WALES, Shrewsbury, MA (US); Andrew James SCHAUBHUT, Bolton, MA (US)

(73) Assignee: Boston Scientific Scimed, Inc., Maple Grove, MN (US)

(21) Appl. No.: 19/291,776

(22) Filed: Aug. 6, 2025

Related U.S. Application Data
(60) Provisional application No. 63/682,512, filed on Aug. 13, 2024.

(51) Int. Cl. A61B 17/00

(52) U.S. Cl. CPC A61B 17/00234; A61B 2017/00292; A61B 2017/0084

(57) ABSTRACT

Medical devices, systems, and related methods described herein may be useful for reducing friction as a portion of a medical device moves during a medical procedure. The medical device including a sheath that defines a lumen therein, a shaft slidably disposed within the lumen, and a covering coupled to an outer surface of the shaft. The covering including a textured outer surface having ridges and channels between the ridges. The textured outer surface configured to decrease friction between the shaft and the sheath during the medical procedure.

### CROSS-REFERENCE TO RELATED APPLICATIONS

This application claims the benefit of priority to U.S. Provisional Application No. 63/682,512, filed on Aug. 13, 2024, which is incorporated by reference herein in its entirety.

### TECHNICAL FIELD

Various aspects of the disclosure relate generally to elements of medical devices, systems, and related methods. Examples of the disclosure relate to member(s) of medical devices having a textured surface for example, to reduce friction between the member(s) and other components of the medical device, among other aspects.

### BACKGROUND

Advancements in medical devices, systems, and related methods, have enabled users to perform increasingly complex medical procedures. Some medical devices may include a shaft or sheath, with one or more wires extending through lumen(s) of the shaft or sheath. For example, a medical device may include a multi-lumen extrusion defining a plurality of lumens. Wire(s) may extend through one or more of the lumens. Conventionally, such devices may include materials such as perfluoroalkyl and polyfluoroalkyl substances (“PFAS”), which may minimize friction between the wire(s) and the lumens. PFAS wires may also be highly resistant to current delivered through the wires.

### SUMMARY

Examples of this disclosure relate to, among other things, medical systems and devices having textured members. For instance, examples of the disclosure relate to medical device and systems having a textured surface that reduces friction as the device moves during medical procedures.

According to one aspect, the disclosure provides a portion of a medical device including a sheath defining a lumen therein, a shaft slidably disposed within the lumen of the sheath, and a covering fixedly coupled to an outer surface of the shaft. The covering includes a textured outer surface including a plurality of ridges and a plurality of channels between the plurality of ridges.

According to some aspects, the covering may include a polymer material, and the shaft may include a metal material. The covering may be formed integrally around the shaft. The covering may be overmolded around the outer surface of the shaft. The textured outer surface may be configured to decrease friction between the shaft and the sheath. The portion of the medical device may include a jacket positioned around at least one portion of the covering. The jacket may include a porous material. The plurality of ridges may extend radially through the jacket.

According to some aspects, the plurality of ridges may have at least one of a fluted profile or a reeded profile. The plurality of ridges may include a first set of ridges having the reeded profile, and a second set of ridges having the fluted profile. Each ridge of the plurality of ridges may extend approximately longitudinally to a central longitudinal axis of the shaft. The plurality of channels may extend approximately parallel to the central longitudinal axis of the shaft. The plurality of channels may be configured to retain a lubricant therein.

According to some aspects, a helical thread may define the plurality of ridges, such that the plurality of ridges is connected to one another. The helical thread may include a first helical thread winding in a first direction, and the covering may include a second helical thread winding in a second direction opposite the first direction.

According to another aspect, the disclosure provides a medical device including a sheath defining a lumen therein, and a member. The member including a shaft disposed within the lumen of the sheath, and a covering fixedly coupled to an outer surface of the shaft, in which the covering comprises a textured outer surface configured to decrease friction as the member moves relative to the sheath. The textured outer surface including a first helical thread defining a first plurality of ridges winding in in a first direction and a first plurality of channels between the first plurality of ridges, and a second helical thread defining a second plurality of ridges winding in a second direction opposite the first direction and a second plurality of channels between the second plurality of ridges.

According to some aspects, the member may include a jacket positioned around at least one portion of the covering, where at least one of the first plurality of ridges and the second plurality of ridges extends radially through the jacket. The shaft may include a metal material, the covering may include a polymer material, and the jacket may include a porous material.

According to yet another aspect, the disclosure provides a medical device including a sheath defining a lumen therein, and a shaft movably disposed within the lumen of the sheath. The outer surface of the shaft including a plurality of scales such that the outer surface has a sharkskin texture configured to decrease friction between the sheath and the shaft. The plurality of scales may be integrally formed with a remainder of the shaft from a same material.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and, together with the description, serve to explain the principles of the disclosure.

FIGS. 1A-1B depict a portion of a medical device, according to aspects of the disclosure.

FIG. 2 depicts an elongate member, according to aspects of the disclosure.

FIG. 3 depicts another elongate member, according to aspects of the disclosure.

FIG. 4 depicts another elongate member, according to aspects of the disclosure.

FIG. 5 depicts another elongate member, according to aspects of the disclosure.

FIG. 6 depicts another elongate member, according to aspects of the disclosure.

FIG. 7 depicts another elongate member, according to aspects of the disclosure.

FIG. 8 depicts another elongate member, according to aspects of the disclosure.

### DETAILED DESCRIPTION

Examples of the disclosure include medical devices having textured surfaces and related methods for facilitating insertion of medical devices having textured surfaces through patient anatomy.

As used herein, the term “distal” refers to a portion farthest away from a user when introducing a device into a patient and the term “proximal” refers to a portion closest to the user when placing the device into the subject. The terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to systems, devices, and methods for performing various medical procedures and/or treating portions of the biliary duct, large intestine, small intestine, cecum, esophagus, any other portion of the gastrointestinal tract, and/or any other suitable patient anatomy (collectively referred to herein as a “target treatment site”). Reference will now be made in detail to examples of the disclosure described above and illustrated in the accompanying drawings. Wherever possible, the same reference numbers will be used throughout the drawings to refer to the same or like parts.

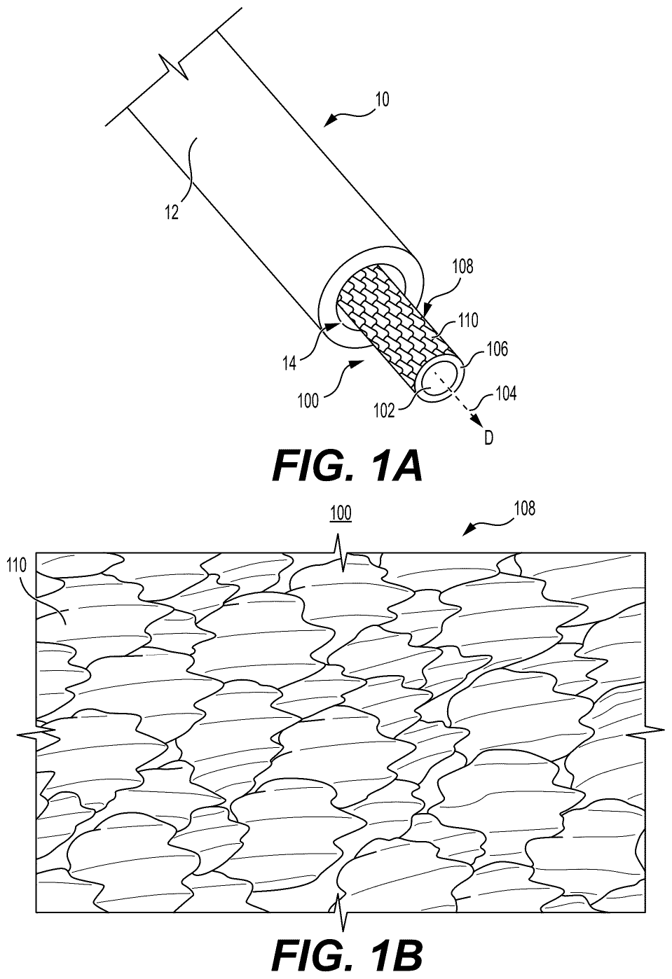

FIG. 1A shows a portion of a medical device 10 including a sheath 12 defining a lumen 14 therein, and an elongate member 100 disposed within lumen 14, according to aspects of the present disclosure. Although sheath 12 is shown with only one lumen 14 for ease of illustration, sheath 12 may have any suitable number of lumens and may be a multi-lumen extrudate shaft. Elongate member 100 may be movable longitudinally (axially) and/or rotate within lumen 14, relative to sheath 12. For example, elongate member 100 may be slidable within lumen 14. Elongate member 100 may include a shaft 102 having a central longitudinal axis 104 (“axis 104”). Elongate member 100 may include a textured outer surface 108 configured to decrease friction as shaft 102 moves (e.g., translates and/or rotates) relative to sheath 12.

In some examples, elongate member 100 includes a wire. Elongate member 100 may be used to actuate features of medical device 10 (e.g., an end effector or other element at a distal tip of medical device 10). Elongate member 100 may additionally or alternatively be used to steer (e.g., articulate or otherwise deflect) a shaft of medical device 10. In some examples, sheath 12 may define an outer surface of a shaft of medical device 10. Alternatively, sheath 12 may be disposed within another sheath or shaft of medical device 10. Elongate member 100 may have any other alternative function (e.g., may serve as a cutting wire of a tome). In another example, elongate member 100 may be a guidewire. Medical device 10 may optionally be delivered through a working channel or other lumen of an endoscope, duodenoscope, gastroscope, colonoscope, ureteroscope, bronchoscope, cystoscope, sheath, and/or various other delivery systems to a target treatment site of patient anatomy during the medical procedure.

Textured outer surface 108 may include one or more patterns or textures. Textured outer surface 108 may define one or more voids configured to, for example, retain lubricant therein. The lubricant reduces friction of elongate member 100 moving (e.g., translating and/or rotating) through lumen 14 of sheath 12 (e.g., during medical procedures). In some examples, the lubricant includes water or a water-based lubricant. In alternatives, a lubricant may be omitted. The plurality of voids defined by textured outer surface 108 may reduce surface area contact between elongate member 100 and an inner surface of sheath 12 (e.g., relative to elongate members without textured outer surface 108). This reduced contact area between elongate member 100 and the inner surface of sheath 12 may reduce friction between elongate member 100 and sheath 12, thereby increasing movability between elongate member and sheath 12.

FIG. 1B depicts a zoomed-in perspective view of textured outer surface 108 of elongate member 100. As shown, textured outer surface 108 may include a plurality of fins or scales 110 defining a scaled surface, similar to a sharkskin. The plurality of scales 110 may optionally be flexible and bend against an inner surface of sheath 12. The plurality of scales 110 may extend at one or more non-zero angles relative to axis 104 of shaft 102. In some examples, at least one of the plurality of scales 110 extends at an acute angle relative to axis 104. In some examples, at least one of scales 110 may extend at a distinct angle relative to another one of scales 110. All of scales 110 may extend in the same general direction (e.g., proximally, or distally). It should be understood that textured outer surface 108 may have different patterns, textures, or configurations to reduce friction.

Scales 110 may extend around an entire circumference of elongate member 100 or at least partially around a circumference of elongate member 100. In some examples, scales 110 may be arranged along an entire length of shaft 102. In other examples, shaft 102 longitudinally extends a first length, and scales 110 cover a second length less than the first length of shaft 102 (i.e., scales 110 only cover a partial longitudinal length of elongate member 100). As further discussed herein, scales 110 may also be implemented in other textured outer surface embodiments.

In examples, outer surface 108 may be formed from the same material as a remainder of elongate member 100 (e.g., shaft 102), such as Nitinol (nickel titanium alloy) or any other suitable material. In other words, outer surface 108 may be integrally, monolithically formed with shaft 102. For example, textured outer surface 108 may be formed by additive manufacturing that includes depositing one or more materials over shaft 102. For example, at least one additive manufacturing process may deposit material on shaft 102 at various locations to form scales, flakes, or other protrusions extending at least partially radially outward from the outer surface of shaft 102. It should be understood that various additive manufacturing processes may be implemented alone or in combination to form the shape and contour of outer surface 108, such as material extrusion (e.g., 3D printing), material jetting, powder bed fusion, binder jetting, directed-energy deposition, stereo lithography, etc. Alternatively, stretched deformation may form outer surface 108. In some examples, deposited material, or a material of shaft 102, may be shaped, polished, or otherwise refined using one or more processes to yield outer surface 108 (e.g., via subtractive manufacturing). In some examples, at least one subtractive manufacturing process removes material from covering 106 to define textured outer surface 108. For example, by engraving one or more portions on the outermost surface of covering 106 to define textured outer surface 108. It should be understood that various subtractive manufacturing processes may be implemented alone or in combination to form the shape and contour of outer surface 108, such as computer numerical control (CNC) machining, electrical discharge machining (EDM), laser cutting, water jet cutting, etc.

In some examples, shaft 102 of elongate member 100 includes a first material, and textured outer surface 108 of elongate member 100 includes a second material (e.g., a coating) different than the first material. In some examples, shaft 102 may include a metal material, such as nickel-titanium alloy or Nitinol. In some examples, outer surface 108 may include a polymer material and/or a porous material. Outer surface 108 made of a different material may be manufactured using any of the techniques described above (e.g., additive manufacturing or subtractive manufacturing). Outer surface 108 may also be overmolded onto shaft 102. In alternative implementations, covering 106 defines a lumen configured to receive shaft 102 therein, and secured to shaft 102 using one or more fixation processes (e.g., adhesive bonding).

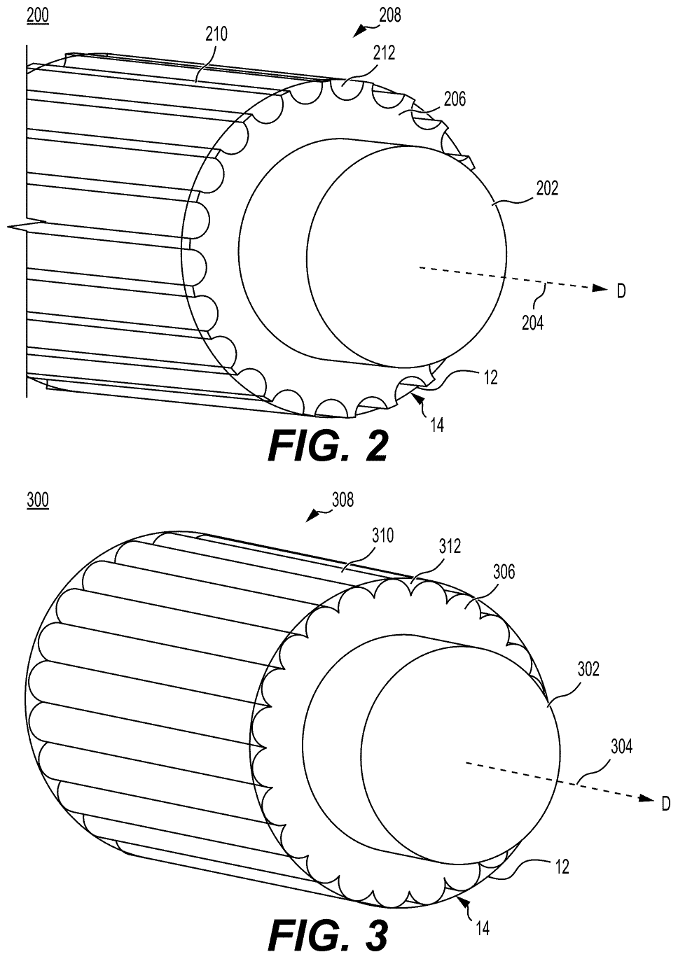

FIG. 2 illustrates another elongate member 200, according to aspects of the disclosure. Unless otherwise specified, aspects of elongate member 200 may be similar to aspects of elongate member 100 (FIG. 1A), the details of which are omitted for brevity.

As shown in FIG. 2, elongate member 200 includes a shaft 202 extending along a central longitudinal axis 204 (“axis 204”), and a covering 206 fixedly coupled to an outer surface of shaft 202. Covering 206 includes a textured outer surface 208 configured to decrease friction as shaft 202 moves during medical procedures. For example, textured outer surface 208 may be useful to reduce friction between elongate member 200 and surfaces of sheath 12 defining lumen 14 that receives elongate member 200 therein.

As shown in FIG. 2, textured outer surface 208 includes a plurality of ridges 210 having a fluted profile. The ridges 210 extending longitudinally along textured outer surface 208 approximately parallel to axis 204. The ridges 210 at least partially define a plurality of channels 212 extending longitudinally along textured outer surface 208 approximately parallel to axis 204 between adjacent ridges 210. The plurality of channels 212 configured to retain lubricant or other fluids (e.g., bodily fluids from a subject) therein, for example to reduce friction as elongate member 200 moves during medical procedures (e.g., through and/or relative to sheath 12 and lumen 14).

As shown in FIG. 2, channels 212 may have surfaces that are curved. For example, surfaces of channels 212 may be approximately semi-circular in cross-section (perpendicular to axis 204). Alternatively, cross-sectional shapes of channels 212 may be other proportions of circles or may be portions of ellipses, parabolas, arcs, or other curved shape. Ridges 210 may have radially outer edges that are substantially flat (e.g., fillet). For example, in cross-section (perpendicular to axis 204), a radially outer surface of each ridge 210 may be approximately linear. Alternatively, ridges 210 may have a curved (e.g., convex or concave) radially outer surface. For example, ridges 210 may have convex radially outer surfaces, such that a circle may be drawn that extends along the radially outer surfaces of all of ridges 210. In another example, ridges 210 may have radially outer edges defining a plurality of sharp edges (e.g., arris) formed by adjacent curved surfaces of channels 212.

Covering 206 may include a same material as shaft 202 or may include a different material (e.g., a polymer or other non-metal). Covering 206 may be formed by additive manufacturing, molding, or subtractive manufacturing, using any of the techniques described above. Covering 206 may include the same material as shaft 202, such that covering 206 may be formed integral or monolithic with shaft 202. In some examples, covering 206 may be overmolded onto shaft 202. In other examples, covering 206 may be separately formed and may be positioned over shaft 202 and secured to shaft 202 (e.g., via adhesive or a friction fit).

As compared with an elongate member lacking covering 206, elongate member 200 may have an overall larger outer surface area, due to ridges 210 and channels 212. This larger surface area encourages lubricant (e.g., water or another fluid) to move along with elongate member 200 because of channels 212. As compared with an elongate member lacking covering 206, elongate member 200 may also have smaller overall contact area with an inner surface of sheath 12 defining lumen 14, in which elongate member 200 is received. For example, only surfaces of ridges 210 may contact inner surfaces of sheath 12, thereby decreasing friction between elongate member 200 and sheath 12 or another shaft or sheath through which elongate member 200 is received.

FIG. 3 illustrates another elongate member 300, according to aspects of the disclosure Unless otherwise specified, aspects of elongate member 300 may be similar to aspects of elongate member 100 (FIG. 1A) and/or elongate member 200 (FIG. 2), the details of which are omitted for brevity.

As shown, elongate member 300 includes a shaft 302 extending along a central longitudinal axis 304 (“axis 304”), and a covering 306 fixedly coupled to an outer surface of shaft 302. Covering 306 includes a textured outer surface 308 configured to decrease friction as shaft 302 moves during medical procedures. For example, textured outer surface 308 may be useful to reduce friction between elongate member 300 and sheath 12.

As shown in FIG. 3, textured outer surface 308 includes a plurality of ridges 310 having a reeded profile. The ridges 310 extend substantially longitudinally along textured outer surface 308 approximately parallel to axis 304. The ridges 310 at least partially define a plurality of channels 312 extending longitudinally along textured outer surface 308 approximately parallel to axis 304. Each of channels 312 may be between adjacent ridges 310 and configured to retain lubricant therein, for example to reduce friction as elongate member 300 moves during medical procedures (e.g., through and/or relative to sheath 12 or lumen 14).

Each of ridges 310 may have a curved, convex outer surface. For example, in cross-section (perpendicular to axis 304), each of ridges 310 may have a semi-circular outer perimeter. Alternatively, a cross-sectional shape of each of ridges 310 may be another portion of a circle, or may have another shape (e.g., a portion of an oval, a portion of an ellipse, a parabola, or another arc). A curved surface of one ridge 310 may abut (be directly adjacent to) a curved surface of an adjacent ridge 310. Thus, each channel 312 may have a radially inner edge that is approximately linear and lacks any substantial width. In other words, when viewed in cross-section (perpendicular to axis 304), a radially innermost portion of each channel 312 may be a point. Channels 312 may be defined by the curved surfaces of adjacent ridges 310.

As compared with an elongate member lacking covering 306, elongate member 300 may have an overall larger outer surface area, due to ridges 310 and channels 312. This larger surface area encourages lubricant (e.g., water or another fluid) to move along with elongate member 300 due to channels 312. As compared with an elongate member lacking covering 306, elongate member 300 may also have smaller overall contact area with an inner surface of sheath 12 in which elongate member 300 is received. For example, only surfaces of ridges 310 may contact inner surfaces of sheath 12 defining lumen 14, thereby decreasing friction between the elongate member 200 and sheath 12. For example, only a line extending along a radially outermost portion of each ridge 310 may contact an inner surface of sheath 12.

Comparing elongate member 200 and 300, channels 212 may have a larger volume than channels 312, which may accommodate a greater volume of lubricant. Ridges 310 may allow for smoother contact with sheath 12 as compared with ridges 210, because of a curved, convex shape of ridges 310. The shape and contour of the curved surfaces defining channels 212 and channels 312 may influence drag coefficients and fluid dynamic drag experienced by elongate member 200 and 300 as each respectively moves through and relative to sheath 12.

In alternative implementations, a textured outer surface 208 or 308 of covering 206 or 306 may include a plurality of ridges including a first set of ridges having the reeded profile of ridges 310 (FIG. 3), and a second set of ridges having the fluted profile of ridges 210 (FIG. 2). For example, the first set of ridges having the reeded profile of ridges 310 may be positioned around a first half of shaft 202 or 302, and the second set of ridges having the fluted profile of ridges 210 may be positioned around a second half of shaft 202 or 302. Alternatively, ridges having the profile of ridges 210 may alternate with ridges of the profile of ridges 310.

In another implementation, one or more ridges may have the fluted profile of ridges 210 and/or the reeded profile of ridges 310 may include one or more exterior portions having the sharkskin profile of scales 110 (FIGS. 1A-1B). For example, at least one of the first set of ridges may have the fluted profile of ridges 210 and may have scales 110 arranged positioned on at least one concave exterior surface.

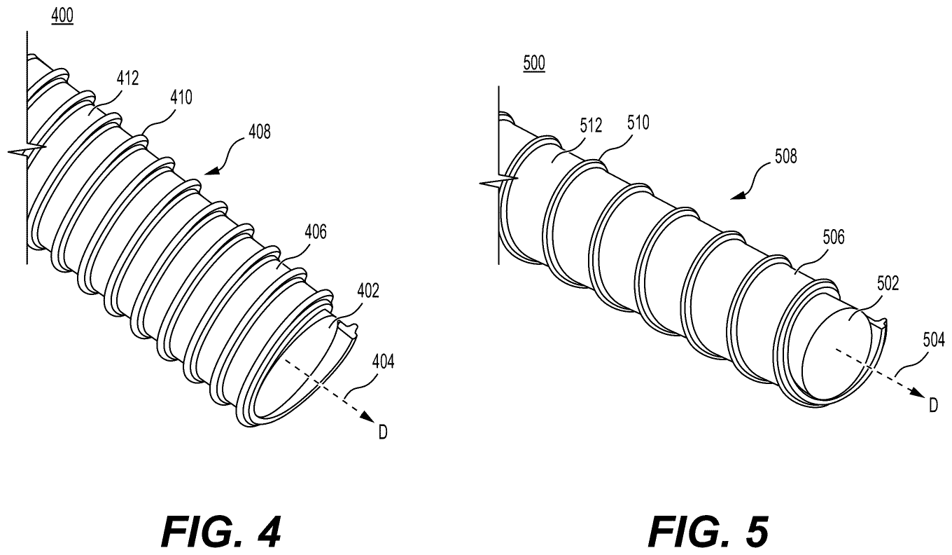

FIG. 4 illustrates an elongate member 400, according to aspects of the disclosure. Unless otherwise specified, aspects of elongate member 400 may be similar to one or more aspects of elongate member 100 (FIG. 1A), elongate member 200 (FIG. 2), and/or elongate member 300 (FIG. 3), details of which are omitted for brevity.

As shown, elongate member 400 includes a shaft 402 extending along a central longitudinal axis 404 (“axis 404”), and a covering 406 fixedly coupled to an outer surface of shaft 402. Covering 406 includes a textured outer surface 408 configured to decrease friction as shaft 402 moves during medical procedures. For example, textured outer surface 408 may be useful to reduce friction between elongate member 400 and a sheath or lumen (such as sheath 12 defining lumen 14) that receives elongate member 400 therein.

Textured outer surface 408 includes a helical thread 410 extending along and about shaft 402. Helical thread 410 has a thread pitch and a thread angle that define a thread profile winding shaft 402 clockwise and/or counter-clockwise relative to axis 404 of shaft 402. Helical thread 410 may define at least one channel 412 of textured outer surface 408 configured to retain lubricant therein, for example to reduce sliding friction as elongate member 400 moves during medical procedures. For example, channels 412 may be defined between adjacent windings of helical thread 410. In some examples, all of channels 412 may be in fluid communication with one another and may be defined as a single channel 412.

In an alternative, thread 410 may include a plurality of discrete rings that extend perpendicularly to axis 404 or at another angle with respect to axis 404. For example, rings of thread 410 may extend partially proximally/distally along axis 404. In such examples, channels 412 may be discrete from one another, separated by the rings of thread 410.

In some aspects, textured outer surface 408 reduces contact surface area between elongate member 400 and the sheath or lumen (not shown) that slidably receives elongate member 400 therein. For instance, helical thread 410 may contact an inner surface of a heath defining a lumen, and the channel(s) is recessed relative to helical thread 410, such that the inner surface of the lumen does not contact the channel(s) as elongate member 400 moves (longitudinally and/or rotationally) therein.

In some aspects, modifying the thread pitch and/or thread angle of helical thread 410 may increase or decrease surface area contacts between covering 406 and the sheath or lumen during medical procedures, and which may correspond to increase or decrease in friction therebetween.

FIG. 5 illustrates an elongate member 500, according to aspects of the disclosure. Unless otherwise specified, aspects of elongate member 500 may be similar to aspects of elongate member 100 (FIG. 1A), elongate member 200 (FIG. 2), elongate member 300 (FIG. 3), and/or elongate member 400 (FIG. 4), details of which are omitted for brevity. In particular, elongate member 500 may have any of the features of elongate member 400, unless otherwise specified.

As shown, elongate member 500 includes a shaft 502 extending along a central longitudinal axis 504, and a covering 506 fixedly coupled to an outer surface of shaft 502. Covering 506 includes a textured outer surface 508 configured to decrease friction while shaft 502 moves during medical procedure. For example, textured outer surface 508 may be useful to reduce sliding friction between elongate member 500 and a sheath or lumen (not shown) that receives elongate member 500 therein. Textured outer surface 508 includes a helical thread 510 having a thread pitch and/or thread angle different than the thread pitch and/or thread angle of textured outer surface 408 (FIG. 4).

For example, as compared with thread 410, thread 510 may have a relatively larger pitch, creating channels 512 with a larger longitudinal length than channels 412. Thread 510 may also have a steeper thread angle, meaning that each winding of thread 510 has an angle that is closer to axis D as compared to thread 410. As compared with elongate member 400, elongate member 500 may constrain lubricant in channels 512 less than lubricant is constrained by channels 412. An overall contact area between elongate member 500 and a sheath surrounding elongate member 500 may be smaller than for elongate member 400, because threads 510 are less dense than threads 410. In some examples, where threads 410 or 510 are very loosely wound, portions of covering 406 or 506 between threads 410 or 510 may also contact an inner surface of the sheath.

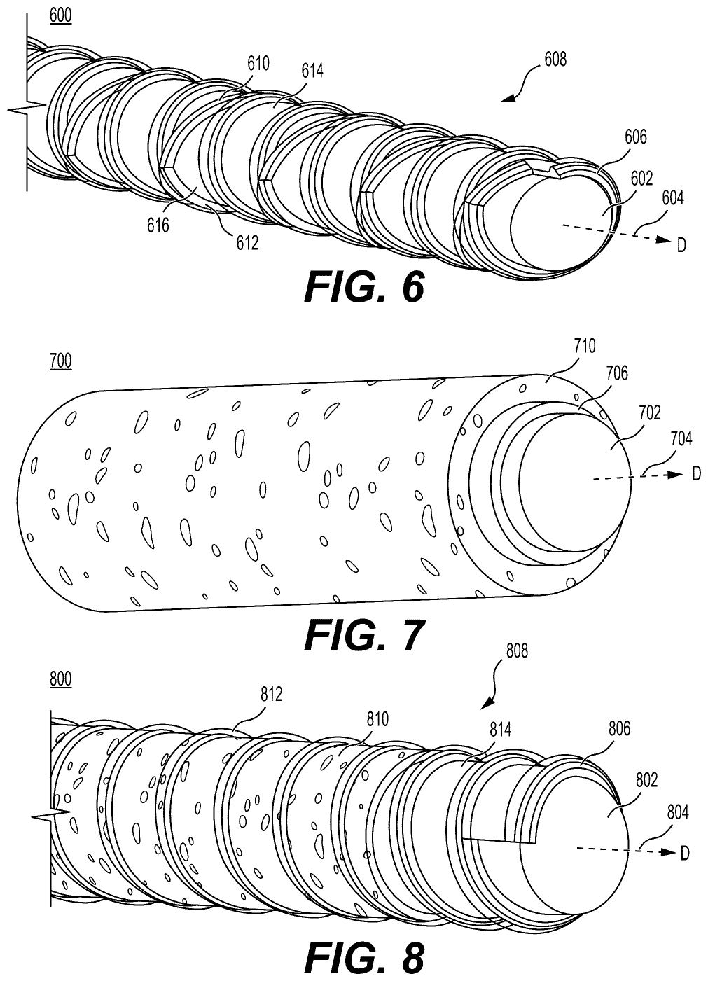

FIG. 6 illustrates an elongate member 600, according to aspects of the disclosure. Unless otherwise specified, aspects of elongate member 600 may be similar to aspects of elongate member 100 (FIG. 1A), elongate member 200 (FIG. 2), elongate member 300 (FIG. 3), elongate member 400 (FIG. 4), and/or elongate member 500 (FIG. 5), details of which are omitted for brevity.

As shown, elongate member 600 includes a shaft 602 extending along a central longitudinal axis 604, and a covering 606 fixedly coupled to an outer surface of shaft 602. Covering 606 includes a textured outer surface 608 configured to decrease friction as shaft 602 moves during medical procedures. For example, textured outer surface 608 may be useful to reduce friction between elongate member 600 and a sheath or lumen (e.g., sheath 12 or lumen 14) that receives elongate member 600 therein.

As shown in FIG. 6, textured outer surface 608 includes a first helical thread 610 winding in a first direction (e.g., clockwise) and a second helical thread 612 winding in a second direction opposite the first direction (e.g., counter-clockwise). First helical thread 610 having a first thread pitch and a first thread angle. Second helical thread 612 having a second thread pitch and a second thread angle.

In some examples, the first thread pitch of first helical thread 610 is the same as the second thread pitch of second helical thread 612, and/or the first thread angle of first helical thread 610 is the same as the second thread angle of second helical thread 612. In other examples, the first thread pitch of first helical thread 610 is the different than the second thread pitch of second helical thread 612, and/or the first thread angle of first helical thread 610 is different than the second thread angle of second helical thread 612.

First helical thread 610 and/or second helical thread 612 may have variable shapes and dimensions to adjust at least one property of textured outer surface 608. Adjusting the thread pitch and/or thread angle changes the shape and dimension of textured outer surface 608, which may be useful to adjust (e.g., increase or decrease) friction of elongate member 600 during medical procedures. For example, increasing thread pitch may increase volume of lubricant that can be loaded in textured outer surface 608, and thus may decrease friction as shaft 602 moves (e.g., translates and/or rotates) during medical procedures (e.g., relative to a sheath or lumen that slidably receives elongate member 600, such as lumen 14 defined by sheath 12 as shown in FIG. 1A).

In some aspects, first helical thread 610 may define a first plurality of ridges that are at least partially interconnected and winding in a first direction, and a first plurality of channels 614 between the first ridges. Second helical thread 612 may define a second plurality of ridges that are at least partially interconnected and winding in a second direction different than the first direction, and a second plurality of channels 616 between the second ridges. In some examples, first helical thread 610 and second helical thread 612 intersect, such that at least one of channels 614 and one of channels 616 at least partially intersect.

FIG. 7 illustrates an elongate member 700, according to aspects of the disclosure. Unless otherwise specified, aspects of elongate member 700 may be similar to aspects of elongate member 100 (FIG. 1A), elongate member 200 (FIG. 2), elongate member 300 (FIG. 3), elongate member 400 (FIG. 4), elongate member 500 (FIG. 5), and/or elongate member 600 (FIG. 6), details of which are omitted for brevity.

As shown, elongate member 700 includes a shaft 702 extending along a central longitudinal axis 704, and a covering 706 fixedly coupled to an outer surface of shaft 702. Elongate member 700 includes a jacket 710 fixedly coupled to one or more portions of covering 706, which is configured to decrease friction as shaft 702 moves during medical procedures. For example, jacket 710 may be useful to retain and release lubricant and thus reduce friction between elongate member 700 and a sheath or lumen (not shown but having any of the properties of sheath 12 and lumen 14) that receives elongate member 700 therein.

Jacket 710 may include a porous material having a plurality of pores configured to load one or more lubricants therein. For example, jacket 710 may include sponge material configured to load lubricant therein (e.g., water or water-based lubricant). During medical procedures, jacket 710 may compress radially inward toward shaft 702 (e.g., due to contact with a sheath or lumen that slidably receives elongate member 700 therein), and in turn causing jacket 710 to release lubricant loaded therein. The lubricant released from jacket 710 decreases sliding friction as the elongate member 700 moves during medical procedures (e.g., relative to a sheath or lumen that slidably receives elongate member 700, such as lumen 14 defined by sheath 12).

Features of two or more elongate members discussed herein may be partially or wholly combined. In some examples, a covering may include a textured outer surface having features from two or more of textured outer surface 108, 208, 308, 408, 508, 608 as discussed herein with reference to FIGS. 1A-6, respectively.

FIG. 8 illustrates an elongate member 800, according to aspects of the disclosure. Unless otherwise specified, aspects of elongate member 800 may be similar to aspects of elongate member 100 (FIG. 1A), elongate member 200 (FIG. 2), elongate member 300 (FIG. 3), elongate member 400 (FIG. 4), elongate member 500 (FIG. 5), elongate member 600 (FIG. 6), and/or elongate member 700 (FIG. 7), details of which are omitted for brevity. In particular, elongate member 800 may have features of elongate member 700 and elongate member 400.

As shown, elongate member 800 includes a shaft 802 extending along a central longitudinal axis 804 (“axis 804”), a covering 806 fixedly coupled to an outer surface of shaft 802 that includes a textured outer surface 808, and a jacket 810 fixedly coupled around one or more portions of covering 806. Textured outer surface 808 includes at least one helical thread 812 winding in at least one direction (e.g., clockwise, and/or counter-clockwise) about covering 806 relative to axis 804. Thread 812 may have any of the features of threads 410, 510.

In some examples, at least one helical thread 812 of textured outer surface 808 may include a first helical thread winding in a first direction and a second helical thread winding in a second direction (e.g., such as first helical thread 610 and second helical thread 612 as shown and discussed with reference to FIG. 6).

In some examples, helical thread 812 of textured outer surface 808 may extend radially through an outer wall of jacket 810. Jacket 810 is substantially similar or identical to jacket 710 as shown and discussed with reference to FIG. 7. Jacket 810 providing porous material positioned in at least one channel defined by textured outer surface 808. For example, helical thread 812 may extend at least partially into or entirely through jacket 810, defining at least one channel 814 having the porous material of jacket 810 disposed therein. Thread pitch and thread angle of helical thread 812 determines a volume of channel 814 and thus determines a volume of porous material of jacket 810 positioned within the volume of channel 814. In some examples, helical thread 812 may extend radially through an entire circumferential thickness of jacket 810. In other examples, helical thread 812 may extend through a portion of the circumferential thickness of jacket 810, such that helical thread 812 is circumferentially surrounded by jacket 810.

In some examples, jacket 810 and/or covering 806 extends longitudinally along an entire extent of shaft 802. In other examples, at least one of jacket 810 and covering 806 extends longitudinally along an extent of shaft 802 less than the entire extent of shaft 802. In one example, jacket 810 extends along a proximal portion of shaft 802 and a distal portion of shaft 802 extends distally beyond a distal end of jacket 810. In another example, covering 806 extends along the proximal portion of shaft 802 and the distal portion of shaft 802 extends distally beyond a distal end of covering 806.

Jacket 810 may include porous material, for example sponge material configured to retain lubricant or other fluids therein. Jacket 810 may include compressible material configured to release lubricant in response to surface area contact (e.g., with inner surface of sheath 12 defining lumen 14 which receives elongate member 800 therein). As elongate member 800 moves (e.g., translates and/or rotates) through and relative to sheath 12, for example, this may cause jacket 810 to compress radially inward relative to shaft 802, and in turn cause jacket 810 to release lubricant loaded therein. This lubricant may reduce friction and facilitate continued movement of elongate member 800 relative to sheath 12.

In some examples, any of the shafts, coverings, or jackets disclosed herein may include one or more tapered portions along the extent thereof. For example, any of the shafts disclosed herein may have a tapered proximal end or a tapered distal end. The coverings or jackets may also taper at their proximal or distal ends. The coverings or jackets may have a variable thickness, such that the radial thickness the jackets or coverings increases and decreases at various locations along the longitudinal extent of the jackets or coverings. In some examples, any of the shafts disclosed herein may extend distally beyond a distal end of their respective coverings or jackets.

Each of the aforementioned embodiments may be used for inserting medical devices into patient anatomy during medical procedures. By providing a medical device having a textured surface configured to reduce friction as the medical device moves relative to a sheath or lumen that slidably receives the medical device therein, known problems associated with invasive surgical procedures may be reduced or avoided. For example, a medical device having textured outer surfaces described herein may reduce sliding friction while moving the medical device through a lumen of a sheath, which may may reduce surface area contact between the device and sheath and/or allow the device to load lubricant therein. Based on these aspects, physicians, or other users of may reduce the overall procedure time, increase efficiency of procedures, and/or avoid unnecessary harm to a patient's body during medical procedures that involve inserting medical devices.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A portion of a medical device, comprising:
a sheath defining a lumen therein;
a shaft slidably disposed within the lumen of the sheath; and
a covering fixedly coupled to an outer surface of the shaft, wherein the covering comprises a textured outer surface including a plurality of ridges and a plurality of channels between the plurality of ridges.

2. The portion of the medical device of claim 1, wherein the plurality of ridges has at least one of a fluted profile or a reeded profile.

3. The portion of the medical device of claim 2, wherein the plurality of ridges comprises a first set of ridges having the reeded profile, and a second set of ridges having the fluted profile.

4. The portion of the medical device of claim 1, wherein the plurality of channels extends approximately parallel to a central longitudinal axis of the shaft.

5. The portion of the medical device of claim 4, wherein each ridge of the plurality of ridges extends approximately longitudinally to a central longitudinal axis of the shaft.

6. The portion of the medical device of claim 1, wherein a helical thread defines the plurality of ridges, such that the plurality of ridges is connected to one another.

7. The portion of the medical device of claim 6, wherein the helical thread is a first helical thread winding in a first direction, and wherein the covering includes a second helical thread winding in a second direction opposite the first direction.

8. The portion of the medical device of claim 1, further comprising a jacket positioned around at least one portion of the covering.

9. The portion of the medical device of claim 8, wherein the jacket comprises a porous material.

10. The portion of the medical device of claim 8, wherein the plurality of ridges extends radially through the jacket.

11. The portion of the medical device of claim 1, wherein the textured outer surface is configured to decrease friction between the shaft and the sheath.

12. The portion of the medical device of claim 1, wherein the plurality of channels is configured to retain a lubricant therein.

13. The portion of the medical device of claim 1, wherein the covering comprises a polymer material, and wherein the shaft comprises a metal material.

14. The portion of the medical device of claim 1, wherein the covering is formed integrally around the shaft.

15. The portion of the medical device of claim 1, wherein the covering is overmolded around the outer surface of the shaft.

16. A medical device, comprising:
a sheath defining a lumen therein; and
a member, including:
a shaft disposed within the lumen of the sheath; and
a covering fixedly coupled to an outer surface of the shaft,
wherein the covering comprises a textured outer surface configured to decrease friction as the member moves relative to the sheath,
wherein the textured outer surface comprises:
a first helical thread defining a first plurality of ridges winding in in a first direction and a first plurality of channels between the first plurality of ridges, and
a second helical thread defining a second plurality of ridges winding in a second direction opposite the first direction and a second plurality of channels between the second plurality of ridges.

17. The medical device of claim 16, wherein the member comprises a jacket positioned around at least one portion of the covering, wherein at least one of the first plurality of ridges and the second plurality of ridges extend radially through the jacket.

18. The medical device of claim 17, wherein the shaft comprises a metal material, wherein the covering comprises a polymer material, and wherein the jacket comprises a porous material.

19. A medical device, comprising:
a sheath defining a lumen therein; and
a shaft movably disposed within the lumen of the sheath, wherein an outer surface of the shaft includes a plurality of scales such that the outer surface has a sharkskin texture configured to decrease friction between the sheath and the shaft.

20. The medical device of claim 19, wherein the plurality of scales is integrally formed with a remainder of the shaft from a same material.
